(19) United States
(12) Patent Application Publication
(10) Pub. No.: US 2022/0211650 A1
(43) Pub. Date: Jul. 7, 2022

(54) SUSTAINED RELEASE FORMULATIONS

(71) Applicant: Hoffman Technologies LLC, New York, NY (US)

(72) Inventors: Steven HOFFMAN, Mahwah, NJ (US); John ROTHMAN, Lebanon, NJ (US)

(21) Appl. No.: 17/604,806

(22) PCT Filed: Apr. 17, 2020
(86) PCT No.: PCT/US2020/028624

§ 371 Date: Oct. 19, 2021

Related U.S. Application Data
(60) Provisional application No. 62/836,256, filed on Apr. 19, 2019.

(51) Int. Cl. A61K 31/198; A61K 38/095; A61K 31/197; A61K 31/27; A61K 31/4178; A61K 31/135; A61K 31/138; A61K 31/4525; A61K 31/137

(52) U.S. Cl. CPC A61K 31/198; A61K 38/095; A61K 31/197; A61K 31/27; A61K 31/137; A61K 31/135; A61K 31/138; A61K 31/4525; A61K 31/4178

(57) ABSTRACT

The invention relates to tyrosine hydroxylase inhibitor compositions and methods thereof. Specifically, the invention relates to a sustained release formulation of a tyrosine hydroxylase inhibitor, particularly α-methyl-para-tyrosine.

### CROSS-REFERENCE TO RELATED APPLICATIONS

This application claims priority to U.S. provisional application No. 62/836,256, filed Apr. 19, 2019, the disclosure of which is incorporated herein by reference in its entirety.

### FIELD OF THE INVENTION

The invention relates to tyrosine hydroxylase inhibitor compositions and methods thereof. Specifically, the invention relates to a sustained release formulation of a tyrosine hydroxylase inhibitor, particularly α-methyl-para-tyrosine.

### BACKGROUND OF THE INVENTION

Tyrosine hydroxylase or tyrosine 3-monooxygenase is the enzyme responsible for catalyzing the conversion of the amino acid L-tyrosine to L-3,4-dihydroxyphenylalanine (L-DOPA). It does so using molecular oxygen (O₂), as well as iron (Fe²⁺) and tetrahydrobiopterin as cofactors.

Tyrosine hydroxylase inhibition can lead to a depletion of dopamine and norepinepherine due to the lack of the precursor L-Dopa (L-3,4-dyhydroxyphenylalanine) which is synthesized by tyrosine hydroxylase.

Various tyrosine hydroxylase inhibitors, for example, α-methyl-L-tyrosine (metyrosine) is well known in the art for treating various diseases and disorders. α-methyl-para-tyrosine (also known as α-methyl-DL-tyrosine or AMPT) is currently under development for the treatment of cancer and autism.

Although tyrosine hydroxylase inhibitors are commercially available, those skilled in the art have not developed any sustained release formulation. To date, no sustained release formulation exists for any of the tyrosine hydroxylase inhibitors including α-methyl-para-tyrosine.

Accordingly, there exists a need for sustained release formulations of tyrosine hydroxylase inhibitors.

### SUMMARY OF THE INVENTION

In one aspect, the invention provides a controlled-release pharmaceutical formulation comprising a tyrosine hydroxylase inhibitor. In one example, the controlled-release formulation is a sustained-release formulation. The sustained-release formulation may include a retardant excipient configured to modify a dissolution profile of said tyrosine hydroxylase inhibitor. The Examples of a retardant excipient may include, but not limited to, hydroxypropyl methylcellulose (HPMC), hydroxyethylcellulose, hydroxypropylcellulose (HPC), methylcellulose, ethylcellulose, cellulose acetate butyrate, cellulose acetate phthalate, hydroxypropylmethyl cellulose phthalate, microcrystalline cellulose, corn starch, polyethylene oxide, polyvinyl alcohol (PVA), polyvinylpyrrolidone (PVP), cross-linked PVP, polyvinyl acetate phthalate, polyethylene glycol, zein, poly-DL-lactide-co-glycolide (PLGA), dicalcium phosphate, calcium sulfate, and mixtures thereof. In a particular embodiment, the tyrosine hydroxylase inhibitor is racemic α-methyl-para-tyrosine.

In another aspect, the invention provides a method of making the controlled-release formulation of the invention, the method comprising intermixing the tyrosine hydroxylase inhibitor with an effective amount of an excipient to form a mixture, and configuring the mixture into a unit dosage form.

In yet another aspect, the invention provides a method of treatment comprising administering the controlled-release formulation of the invention to a patient in need thereof.

In certain embodiments, the controlled-release pharmaceutical formulation comprises α-methyl-para-tyrosine, α-methyl-D-tyrosine, α-methyl-L-tyrosine, or a combination thereof, dispersed in a wax matrix.

In various embodiments, the controlled-release pharmaceutical formulation comprises α-methyl-para-tyrosine, α-methyl-D-tyrosine, α-methyl-L-tyrosine, or a combination thereof, dispersed in polymer matrix.

In other embodiments, the controlled-release pharmaceutical formulation comprises α-methyl-para-tyrosine, α-methyl-D-tyrosine, α-methyl-L-tyrosine, or a combination thereof, dispersed in an encapsulated form.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount. Thus, for example, reference to a formulation that comprises “about 70% tyrosine hydroxylase inhibitor by weight” will be understood as a reference to an amount of tyrosine hydroxylase inhibitor in the pharmaceutical formulation that is 70%±14% (i.e., between 56% and 84%) by weight, or preferably 70%±7% (i.e., between 63% and 77% by weight), or more preferably 70%±4% (i.e., between 66% and 74% by weight).

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The term “stereoisomers” refers to compounds that have identical chemical constitution, but differ as regards the arrangement of the atoms or groups in space. The term “enantiomers” refers to stereoisomers that are mirror images of each other that are non-superimposable.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

The term “inhibitor” as used herein includes compounds that inhibit the expression or activity of a protein, polypeptide or enzyme and does not necessarily mean complete inhibition of expression and/or activity. Rather, the inhibition includes inhibition of the expression and/or activity of a protein, polypeptide or enzyme to an extent, and for a time, sufficient to produce the desired effect.

Various embodiments provide pharmaceutical formulations that provide controlled-release of a tyrosine hydroxylase inhibitor. Such formulations can be configured in various ways and in a variety of dosage forms, such as tablets and beads, to modify the release of the tyrosine hydroxylase inhibitor. For example, one type of controlled-release pharmaceutical formulation is a sustained-release tyrosine hydroxylase inhibitor pharmaceutical formulation. Sustained-release tyrosine hydroxylase inhibitor pharmaceutical formulations can contain a variety of excipients, such as retardant excipients (also referred to as release modifiers) and/or fillers that are selected and incorporated into the formulation in such a way as to slow the dissolution rate of the formulation (and thereby slow the dissolution and/or release of the tyrosine hydroxylase inhibitor) under in vivo conditions as compared to an otherwise comparable immediate-release formulation.

The term “immediate-release” is used herein to specify a formulation that is not configured to alter the dissolution profile of the active ingredient (e.g., tyrosine hydroxylase inhibitor). For example, an immediate-release pharmaceutical formulation may be a pharmaceutical formulation that does not contain ingredients that have been included for the purpose of altering the dissolution profile. An immediate-release formulation thus includes drug formulations that take less than 30 minutes for substantially complete dissolution of the drug in a standard dissolution test. A “standard dissolution test,” as that term is used herein, is a test conducted according to United States Pharmacopeia 24th edition (2000) (USP 24), pp. 1941-1943, using Apparatus 2 described therein at a spindle rotation speed of 100 rpm and a dissolution medium of water, at 37° C., or other test conditions substantially equivalent thereto.

The term “controlled-release” is used herein in its ordinary sense and thus includes pharmaceutical formulations that are combined with ingredients to alter their dissolution profile. A “sustained-release” formulation is a type of controlled-release formulation, wherein ingredients have been added to a pharmaceutical formulation such that the dissolution profile of the active ingredient is extended over a longer period of time than that of an otherwise comparable immediate-release formulation. A controlled-release formulation thus includes drug formulations that take 30 minutes or longer for substantially complete dissolution of the drug in a standard dissolution test, conditions which are representative of the in vivo release profile.

The term “orally deliverable” is used herein in its ordinary sense and thus includes drug formulations suitable for oral, including peroral and intra-oral (e.g., sublingual or buccal) administration. Preferred compositions are adapted primarily for peroral administration, e.g., for swallowing. Examples of preferred orally deliverable compositions include discrete solid articles such as tablets and capsules, which are typically swallowed whole or broken, with the aid of water or other drinkable fluid.

The term in vivo “absorption” is used herein in its ordinary sense and thus includes reference to the percentage of tyrosine hydroxylase inhibitor that enters the bloodstream, as conventionally calculated from data of a standard pharmacokinetic (PK) study involving oral administration of a single dose of tyrosine hydroxylase inhibitor. It will be understood that PK data are subject to the usual variation seen in biological data, in accordance with standard statistical practice.

A “subject” herein is an animal of any species, preferably mammalian, most preferably human. Conditions and disorders in a subject for which a particular drug or compound (such as tyrosine hydroxylase inhibitor) is said herein to be “indicated” are not restricted to conditions and disorders for which that drug or compound has been expressly approved by a regulatory authority, but also include other conditions and disorders known or reasonably believed by a physician to be amenable to treatment with that drug or compound.

### Tyrosine Hydroxylase Inhibitor

The tyrosine hydroxylase inhibitor is well known in the art and fully described in, for example, U.S. Patent Application Publications US 2015/0290279, US 2015/0216827, US 2015/0111937, US 2015/0111878, US 2013/0184214, and US 20130183263; U.S. Pat. Nos. 8,481,498, 9,308,188, and 9,326,962; and PCT Patent Application Publication WO2015061328, which are incorporated by reference herein in their entirety. Any suitable tyrosine hydroxylase inhibitor, known to one of skilled in the art, can be used.

In certain embodiments, the tyrosine hydroxylase inhibitor is a tyrosine derivative. The tyrosine derivative can be capable of existing in different isomeric forms, including stereoisomers and enantiomers. The tyrosine derivative can, for example, exist in both L-form or D-form. The tyrosine derivative can, for example, also exist in a racemic form.

Representative tyrosine derivatives include, for example, one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-tyrosine(tBu)-allyl ester hydrochloride, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyrosine methyl ester hydrochloride, H-3,5-diiodo-tyrosine methyl ester hydrochloride, H-D-3,5-diiodo-tyrosine methyl ester hydrochloride, H-D-tyrosine methyl ester hydrochloride, D-tyrosine methyl ester hydrochloride, D-tyrosine-methyl ester hydrochloride, methyl D-tyrosinate hydrochloride, H-D-tyrosine methyl ester″hydrochloride, D-tyrosine methyl ester hydrochloride, H-D-tyrosine methyl ester-hydrochloride, (2R)-2-amino-3-(4-hydroxyphenyl)propionic acid, (2R)-2-amino-3-(4-hydroxyphenyl)methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-tyrosine (3,5-I2)-OSu, Fmoc-tyrosine(3-N02)-OH, α-methyl-L-tyrosine, α-methyl-D-tyrosine, and α-methyl-para-tyrosine. In certain embodiments of the invention, the tyrosine derivative is α-methyl-L-tyrosine as shown below:

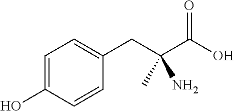

In other embodiments, the tyrosine derivative is α-methyl-D-tyrosine. In other embodiments, the tyrosine derivative is α-methyl-para-tyrosine in a racemic form as shown below:

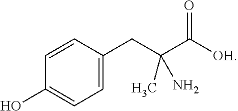

α-methyl-para-tyrosine is also referred herein as DNP-01 or LI:79 or AMPT or α-methyl-DL-tyrosine. In other words, the alternative names of α-methyl-para-tyrosine include, for example, DNP-01, LI:79, AMPT, and α-methyl-DL-tyrosine.

In a particular embodiment, the tyrosine derivative is a structural variant of α-methyl-L-tyrosine or α-methyl-para-tyrosine. The structural variants of α-methyl-L-tyrosine or α-methyl-para-tyrosine are well known in the art and fully described in, for example, U.S. Pat. No. 4,160,835, which is incorporated by reference herein in its entirety.

In one embodiment, the tyrosine derivative of the invention is an arylalanine compound having the formula:

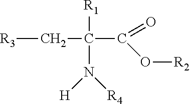

wherein R₁ is hydrogen, methyl or ethyl ester group, or alkyl of from 1 to 4 carbon atoms; R₂ is hydrogen, lower alkyl, lower alkene, succinimide, or alkyl of from 1 to 4 carbon atoms; R₃ is a substituted benzene ring of the following general formula

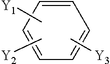

wherein Y₁, is located at the para position and is hydrogen, hydroxy, methyl ether, dimethyl ether, trimethyl ether, or an unsubstituted or halogen-substituted benzyl; Y₂, and Y₃ are the same or different and wherein one or both Y₂, and Y₃ located at either meta position or ortho position, and wherein Y₂, and Y₃ are hydrogen, hydroxy, halogen, methyl ether, or nitro; and R₄ is hydrogen, acetyl, tert-butyloxycarbonyl or fluorenylmethyloxycarbonyl.

In some embodiments, Y1 and Y2 are the same or different and are selected from hydrogen, cyanoamino, carboxyl, cyano, thiocarbamoyl, aminomethyl, guanidino, hydroxy, methanesulfonamido, nitro, amino, methanesulfonyloxy, carboxymethoxy, formyl, methoxy and a substituted or unsubstituted 5- or 6-membered heterocyclic ring containing carbon and one or more nitrogen, sulfur or oxygen atoms, specific examples of such heterocyclic rings being pyrrol-1-yl, 2-carboxypyrrol-1-yl, imidazol-2-ylamino, indol-1-yl, carbazol-9-yl, 4,5-dihydro-4-hydroxy-4-trifluoromethylthiazol-3-yl, 4-trifluoromethylthiazol-2-yl, imidazol-2-yl and 4,5-dihydroimidazol-2-yl, such that (a) Y1 and Y2 cannot both be hydroxy, (b) Y1 and Y2 cannot both be hydrogen and (c) when one of Y1 and Y2 is hydrogen, the other cannot be hydroxyl.

In one example, R₃ is a substituted or unsubstituted benzoheterocyclic ring having the formula:

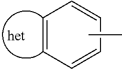

in which the benzoheterocyclic ring is selected from the group consisting of indolin-5-yl, 1-(N-benzoylcarbamimidoyl)-indolin-5-yl, 1-carbamimidoylindolin-5-yl, 1H-2-oxindol-5-yl, indol-5-yl, 2-mercaptobenzimidazol-5(6)-yl, 2-aminobenzimidazol-5(6)-yl, 2-methanesulfonamido-benzimidazol-5(6)-yl, 1H-benzoxazol-2-on-6-yl, 2-aminobenzothiazol-6-yl, 2-amino-4-mercaptobenzothiazol-6-yl, 2,1,3-benzothiadiazol-5-yl, 1,3-dihydro-2,2-dioxo-2,1,3-benzothiadiazol-5-yl, 1,3-dihydro-1,3-dimethyl-2,2-dioxo-2,1,3-benzothiadiazol-5-yl, 4-methyl-2(1H)-oxoquinolin-6-yl, quinoxalin-6-yl, 2-hydroxquinoxalin-6-yl, 2-hydroxyquinoxalin-7-yl, 2,3-dihydroxyquinoxalin-6-yl and 2,3-dihydro-3(4H)-oxo-1,4-benzoxazin-7-yl.

In another example, R₃ is a substituted or unsubstituted heterocyclic ring having the formula:

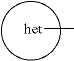

in which the heterocyclic ring is selected from the group consisting of 5-hydroxy-4H-pyran-4-on-2-yl, 2-hydroxypyrid-4-yl, 2-aminopyrid-4-yl, 2-carboxypyrid-4-yl, or tetrazolo[1,5-a]pyrid-7-yl.

In one particular embodiment, the tyrosine hydroxylase inhibitor is aquayamycin. In one example, aquayamycin is a compound of the formula set forth below.

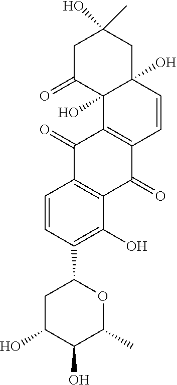 [SMILES: C[C@H]1O[C@@H](c2ccc3c(c2O)C(=O)C2=C(C3=O)[C@@]3(O)C(=O)C[C@](C)(O)C[C@@]3(O)C=C2)C[C@@H](O)[C@@H]1O]

In another particular embodiment, the tyrosine hydroxylase inhibitor is oudenone. In one example, oudenone is a compound of the formula set forth below.

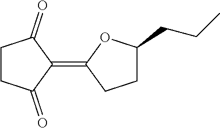

Other suitable tyrosine hydroxylase inhibitor, known to one of skilled in the art, can also be used. Example of other tyrosine hydroxylase inhibitor include, for example, but not limited to, cycloheximide, anisomycin, 3-iodo-L-tyrosine, pyratrione, phenyl carbonyl derivatives having catechol or triphenolic ring systems, for example, phenethylamine and gallic acid derivatives, 4-isopropyltropolone, 2-(4-thiazolyl)benzimidazole, 8-hydroxyquinoline, o-phenantroline, 5-iodo-8-hydroxyquinoline, bilirubin, 2,9-dimethyl-1, 10-phenantroline, α-α′-dipyridil, dibenzo hi quinoxaline, 2,4,6-tripyridil-s-triazine, ethyl 3-amino4H-pyrrolo-isoxazole-5(6H)-carboxylate, α-nitroso-β-naphthol, sodium diethyldithiocarbamate, ethylenediamineteraacetic acid (See R Hochster, Metabolic Inhibitors V4: A Comprehensive Treatise 52 Elsevier (2012)).

### Sustained-Release Formulation

In some embodiments, the sustained-release tyrosine hydroxylase inhibitor pharmaceutical formulation comprises one or more retardant excipients. In this context, the term “retardant” excipient is used herein in its ordinary sense and thus includes an excipient that is configured (e.g., incorporated into the formulation) in such a way as to control a dissolution profile of the drug, e.g., slow the dissolution of the tyrosine hydroxylase inhibitor in a standard dissolution test, as compared to an otherwise comparable pharmaceutical formulation that does not contain the retardant excipient. Examples of pharmaceutically acceptable retardant excipients include, but not limited to, hydroxypropyl methylcellulose (HPMC), hydroxyethylcellulose, hydroxypropylcellulose (HPC), methylcellulose, ethylcellulose, cellulose acetate butyrate, cellulose acetate phthalate, hydroxypropylmethyl cellulose phthalate, microcrystalline cellulose, corn starch, polyethylene oxide, polyvinyl alcohol (PVA), polyvinylpyrrolidone (PVP), cross-linked PVP, polyvinyl acetate phthalate, polyethylene glycol, zein, poly-DL-lactide-co-glycolide, dicalcium phosphate, calcium sulfate, and mixtures thereof. In some embodiments the retardant excipient comprises a sustained-release polymer, e.g., at least one of hydroxypropyl methylcellulose (HPMC), hydroxyethylcellulose, hydroxypropylcellulose (HPC), methylcellulose, ethylcellulose, cellulose acetate butyrate, cellulose acetate phthalate, hydroxypropylmethyl cellulose phthalate, microcrystalline cellulose, corn starch, polyethylene oxide, polyvinyl alcohol (PVA), polyvinylpyrrolidone (PVP), cross-linked PVP, polyvinyl acetate phthalate, polyethylene glycol, zein, poly-DL-lactide-co-glycolide (PLGA), and mixtures thereof. Retardant excipients may be referred to herein as release modifiers.

In certain embodiments, the controlled-release pharmaceutical formulation comprises α-methyl-para-tyrosine, α-methyl-D-tyrosine, α-methyl-L-tyrosine, or a combination thereof, dispersed in a wax matrix.

In some embodiments, the wax matrix comprises a retardant excipient, which is insoluble and erodible in water, including but not limited to, carnauba wax, stearyl alcohol, stearic acid, polyethylene glycol hydrogenated castor oil, castor wax, polyethylene glycol monostearate, and triglycerides.

In various embodiments, the controlled-release pharmaceutical formulation comprises α-methyl-para-tyrosine, α-methyl-D-tyrosine, α-methyl-L-tyrosine, or a combination thereof, dispersed in polymer matrix.

In some embodiments, the polymer matrix comprises a retardant excipient, which is water insoluble and inert in water, including but not limited to, ethyl cellulose, polyethylene, methyl acrylate-methacrylate copolymer, and polyvinyl chloride.

In other embodiments, the polymer matrix comprises a retardant excipient, which is hydrophilic and soluble in water, including but not limited to, cellulose derivatives (including, but not limited to, methylcellulose, hydroxyethyl cellulose, hydroxypropylmethyl cellulose (HPMC), sodium carboxymethyl cellulose (“sodium CMC”); non-cellulose polysaccharides (including, but not limited to sodium alginate, potassium alginate, agar, carrageen, xanthan gum, arabic gum, and caraia gum; galactomannose, guar gum, alfarroba gum); and acrylic acid polymers (including, but not limited to carboxypolymethylene).

In other embodiments, the controlled-release pharmaceutical formulation comprises α-methyl-DL-tyrosine, α-methyl-D-tyrosine, α-methyl-L-tyrosine, or a combination thereof, dispersed in an encapsulated form.

In a matrix system, the drug is dispersed as solid particle within a porous matrix formed of a water insoluble polymer, such as polyvinyl chloride.

In various embodiments, the matrix system may be a slowly eroding matrix, including but not limited to waxes, glycerides, stearic acid, cellulosic materials. In some embodiments, a portion of the drug intended to have sustained action is combined with lipid or cellulosic material and then granulated.

In certain embodiments, the drug may be embedded in an inert plastic matrix. In embodiments, the drug may be granulated with an inert, insoluble matrix, including but not limited to polyethylene, polyvinyl acetate, polystyrene, polyamide or polymethacrylate.

In certain embodiments, the drug may be coated on its surface with a material, such as with a polymer) that retards penetration by the dispersion fluid. The coating may be performed by microencapsulation, a process in which a relatively thin coating is applied to small particles of solid or droplets of liquids and dispersion. In embodiments, polymers, include but are not limited to, polyvinyl alcohol, polyacrylic acid, ethylcellulose, polyethylene, polymethacrylate, poly(ethylene-vinylacetate), cellulose nitrite, silicones, poly (lactide-co-glycolide).

There are various ways that an excipient can be configured to control a dissolution profile of a sustained-release formulation. For example, the excipient can be intimately mixed with the drug (e.g., tyrosine hydroxylase inhibitor) in an amount effective for controlling release of the drug from the pharmaceutical formulation. Such a mixture can be in various forms, e.g., a dry mixture, a wet mixture, tablet, capsule, beads, etc., and may be formed in various ways. The resulting mixture can then be formed into the desired dosage form, e.g., tablet or capsule.

Effective amounts of retardant excipient(s) for controlling release may be determined by the guidance provided herein. For example, in some embodiments the sustained-release pharmaceutical formulation comprises at least about 5, 10, 15, 20, 25, 30, 35, 40, 45, 50, 55, 60, 65, 70, 75, 80, 85, 90, 95% (w/w) of the retardant excipient(s). In some embodiments, the concentration of the retardant excipient(s) in the pharmaceutical formulation may range about 5-95, 10-80, 20-70, 25-65, 35-55, 40-50, 5-20, 10-30, 20-40, 30-50, 40-60, 50-70, 60-80, 70-95% (w/w).

Various dissolution characteristics of the dissolution profile of the sustained-released tyrosine hydroxylase inhibitor pharmaceutical formulation can be controlled by appropriate configuration of the retardant excipient incorporated therein. Preferably, the dissolution profile comprises a dissolution rate that is slower than a dissolution rate of a comparable immediate-release tyrosine hydroxylase inhibitor formulation. For example, in some embodiments, the pharmaceutical formulation comprises tyrosine hydroxylase inhibitor and at least one retardant excipient configured to control an in vitro release profile within the following ranges of drug release: 0-40% released in 1 hour; 10-60% released in 4 hours; 20-80% released in 8 hours; >=70% released in 12 hours.

In an exemplary embodiment, the sustained-release pharmaceutical formulation comprises tyrosine hydroxylase inhibitor and at least one retardant excipient configured to provide, upon administration to a patient, an average free serum tyrosine hydroxylase inhibitor Cₘₐₓ value that is less than (e.g., at least about 5% less than) the average free serum tyrosine hydroxylase inhibitor Cₘₐₓ value of a comparable immediate-release tyrosine hydroxylase inhibitor under comparable conditions. For example, the retardant excipient can be configured to control an in vivo free tyrosine hydroxylase inhibitor serum profile wherein there is greater tyrosine hydroxylase inhibitor bioavailability, as indicated by an area under the serum concentration curve at steady state that is substantially equal to or greater than a conventional immediate-release tyrosine hydroxylase inhibitor formulation at the same dose, and a lower Cₘₐₓ at steady state than a conventional immediate-release tyrosine hydroxylase inhibitor formulation at the same dose.

Sustained-release tyrosine hydroxylase inhibitor pharmaceutical formulation as described herein may be formulated to be useful for oral administration under dosage schedules in the range of once or twice daily to once every two to seven days, to a subject having a condition or disorder for which the administration of tyrosine hydroxylase inhibitor is indicated. Thus, in some embodiments a pharmaceutical formulation comprises a controlled dosage form suitable for daily or weekly administration of tyrosine hydroxylase inhibitor.

Certain sustained-release tyrosine hydroxylase inhibitor formulations may exhibit one or more surprising and unexpected features and benefits. For example, sustained-release dosage forms are typically sought to enable longer time intervals between dosing of a drug having a short half-life in plasma, due for example to rapid metabolism, excretion or other routes of depletion.

In an embodiment, a method of treatment comprises administering a sustained-release pharmaceutical formulation as described herein to a patient in need thereof.

In some embodiments, the sustained-release pharmaceutical formulation is formed into capsules, tablets or other solid dosage forms suitable for oral administration. In preferred embodiments, the sustained-release pharmaceutical formulation is formulated as a discrete solid dosage unit such as a tablet or capsule, wherein the tyrosine hydroxylase inhibitor or salt thereof is present therein as particles, and is formulated together with one or more pharmaceutically acceptable excipients. In some embodiments the excipients are retardant excipients selected at least in part to provide a release profile and/or PK profile consistent with the desired profiles described herein.

In some embodiments the particular solid dosage form selected is not critical so long as it achieves a release and/or PK profile as defined herein for the particular sustained-release formulation. In some embodiments the profile is achieved using one or more retardant excipients or release modifiers. In some embodiments release modifiers suitable for use include a wax or polymer matrix with which and/or in which the tyrosine hydroxylase inhibitor is dispersed; a release-controlling layer or coating surrounding the whole dosage unit or tyrosine hydroxylase inhibitor-containing particles, granules, beads or zones within the dosage unit.

Sustained-release pharmaceutical formulations can be configured in a variety of dosage forms, such as tablets and beads; can contain a variety of fillers and excipients, such as retardant excipients (also referred to a release modifiers); and may be made in a variety of ways. Those skilled in the art may determine the appropriate configuration by routine experimentation guided by the descriptions provided herein.

Sustained-release pharmaceutical formulations may contain fillers. Examples of suitable fillers include, but are not limited to, METHOCEL, methylcellulose, hydroxypropyl methylcellulose (HPMC), hydroxypropylcellulose (HPC), corn starch, polyvinyl alcohol (PVA), polyvinylpyrrolidone (PVP), cross-linked PVP, and the like.

Sustained-release tyrosine hydroxylase inhibitor pharmaceutical formulations may contain other excipients. Examples of suitable excipients include, but are not limited to, acetyltriethyl citrate (ATEC), acetyltri-n-butyl citrate (ATBC), aspartame, lactose, alginates, calcium carbonate, carbopol, carrageenan, cellulose, cellulose acetate phthalate, croscarmellose sodium, crospovidone, dextrose, dibutyl sebacate, ethylcellulose, fructose, gellan gum, glyceryl behenate, guar gum, lactose, lauryl lactate, low-substituted hydroxypropyl cellulose (L-HPC), magnesium stearate, maltodextrin, maltose, mannitol, methylcellulose, microcrystalline cellulose, methacrylate, sodium carboxymethylcellulose, polyvinyl acetate phthalate (PVAP), povidone, shellac, sodium starch glycolate, sorbitol, starch, sucrose, triacetin, triethylcitrate, vegetable based fatty acid, xanthan gum, xylitol, and the like.

In some embodiments, the sustained-release pharmaceutical formulation comprises, for example, from about 5%, 10%, 20% 30%, 40%, or 50%, to about 60%, 70%, 80%, 90% or 95% tyrosine hydroxylase inhibitor by weight. For example, in some embodiments the sustained-release pharmaceutical formulation comprises at least about 5, 10, 15, 20, 25, 30, 35, 40, 45, 50, 55, 60, 65, 70, 75, 80, 85, 90, 95% (w/w) of tyrosine hydroxylase inhibitor. In some embodiments, the concentration of tyrosine hydroxylase inhibitor in the pharmaceutical formulation may range about 5-95, 10-80, 20-70, 25-65, 35-55, 40-50, 5-20, 10-30, 20-40, 30-50, 40-60, 50-70, 60-80, 70-95% (w/w).

The dissolution rate of the sustained-release tyrosine hydroxylase inhibitor pharmaceutical formulation determines how quickly tyrosine hydroxylase inhibitor becomes available for absorption into the blood stream and therefore controls the bioavailability of tyrosine hydroxylase inhibitor. Dissolution rate is dependent on the size and the composition of the dosage form. In some embodiments, the dissolution rate of the tyrosine hydroxylase inhibitor formulation can be by changed by altering the additional components of the formulation. Disintegrants, such as starch or corn starch, or crosslinked PVPs, can be used to increase solubility when desired. Solubilizers can also be used to increase the solubility of the tyrosine hydroxylase inhibitor formulations. In some embodiments alternative binders, such as hydroxypropylmethyl cellulose (HPMC), hydroxypropyl cellulose (HPC), methyl cellulose (MC), PVP, gums, xanthine, and the like, can be used to increase the dissolution rate.

In some embodiments the dissolution rate of the formulation can be decreased by adding components that make the formulation more hydrophobic. For example, addition of polymers such as ethylcelluloses, wax, magnesium stearate, and the like can decrease the dissolution rate.

In some embodiments, the dissolution rate of the sustained-release pharmaceutical formulation is such that about 25% of the tyrosine hydroxylase inhibitor in the dosage form is dissolved within the first hour, about 60% of the tyrosine hydroxylase inhibitor is dissolved within the first 6 hours, about 80% of the tyrosine hydroxylase inhibitor is dissolved within the first 9 hours, and substantially all of the tyrosine hydroxylase inhibitor is dissolved within the first 12 hours. In other embodiments, the dissolution rate of the sustained-release pharmaceutical formulation is such that about 35% of the tyrosine hydroxylase inhibitor in the dosage form is dissolved within the first hour, about 85% of the tyrosine hydroxylase inhibitor is dissolved within the first 6 hours, and substantially all of the tyrosine hydroxylase inhibitor is dissolved within the first 9 hours. In yet other embodiments, the dissolution rate of the sustained-release pharmaceutical formulation in the dosage form is such that about 45% of the tyrosine hydroxylase inhibitor is dissolved within the first hour, and substantially all of the tyrosine hydroxylase inhibitor is dissolved within the first 6 hours.

The dissolution rate of the formulation can also be slowed by coating the dosage form. Examples of coatings include enteric coatings, sustained-release polymers, and the like.

The sustained-release pharmaceutical formulation can take about, for example, from 2, 4, 6, or 8 hours to about 15, 20, or 25 hours to dissolve. Preferably, the formulation has a dissolution rate of from about 3, 4, 5, or 6 to about 8, 9, or 10 hours.

Another embodiment provides a method of preparing sustained-release pharmaceutical formulations. The method comprises mixing tyrosine hydroxylase inhibitor with an excipient and/or filler to form a mixture, and forming a suitable dosage form (e.g., tablet, capsule, bead, etc.) from the mixture. In some embodiments, the method of preparing the formulation further comprises adding another excipient and/or filler to the mixture prior to forming the dosage form. The filler and excipient are as described herein. In an embodiment, the tyrosine hydroxylase inhibitor is mixed with the filler and/or excipient to form a wet mixture. The wet mixture can then be formed into particles or beads, which can then be dried. The dried product can then be tableted or placed into a gelatin capsule for oral delivery.

In an embodiment, a pharmaceutical formulation comprises a sustained-release tyrosine hydroxylase inhibitor and a filler. In some embodiments the formulation further comprises an excipient. In some embodiments the filler is a polymer. In some embodiments the excipient is a polymer. In some embodiments the filler is selected from the group consisting of methylcellulose, hydroxypropyl methylcellulose (HPMC), hydroxypropylcellulose (HPC), corn starch, polyvinyl alcohol (PVA), polyvinylpyrrolidone (PVP), and cross-linked PVP. In some embodiments the excipient is selected from the group consisting of acetyltriethyl citrate (ATEC), acetyltri-n-butyl citrate (ATBC), aspartame, lactose, alginates, calcium carbonate, carbopol, carrageenan, cellulose, cellulose acetate phthalate, croscarmellose sodium, crospovidone, dextrose, dibutyl sebacate, ethylcellulose, fructose, gellan gum, glyceryl behenate, guar gum, lactose, lauryl lactate, low-substituted hydroxypropyl cellulose (L-HPC), magnesium stearate, maltodextrin, maltose, mannitol, methylcellulose, microcrystalline cellulose, methacrylate, sodium carboxymethylcellulose, polyvinyl acetate phathalate (PVAP), povidone, shellac, sodium starch glycolate, sorbitol, starch, sucrose, triacetin, triethylcitrate, vegetable based fatty acid, xanthan gum, and xylitol.

The invention also provides a pharmaceutical composition comprising compounds of the invention and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or additional therapeutic agents.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Pharmaceutically acceptable carriers include solvents, dispersion media, buffers, coatings, antibacterial and antifungal agents, wetting agents, preservatives, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

During the manufacturing, the carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Effective doses of the compositions of the present invention, for treatment of conditions or diseases vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

The pharmaceutical compositions of the invention may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

In one aspect, the dosage of tyrosine hydroxylase inhibitor may range from about 1 mg to about 4 g. In a particular embodiment, the dosage of tyrosine hydroxylase inhibitor may range from about 3 mg to about 1000 mg. In some suitable embodiments the drug is given in divided doses. In some suitable embodiments of the invention, 25 mg of the tyrosine hydroxylase inhibitor is administered. In one example, 60 mg of the tyrosine derivative can be administered.

In another aspect, the dosage of another agent useful in the treatment of a disease may include a therapeutically effective or clinically acceptable amount. In another example, the dosage of another agent is an amount that complements with or enhances the effect of a tyrosine hydroxylase inhibitor described herein.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The composition of the invention may be administered only once, or it may be administered multiple times. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated, or the form of sustained release technology employed. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

“Administration” to a subject is not limited to any particular delivery system and may include, without limitation, oral administration (for example, in capsules or tablets). Administration to a host may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.).

Patient compliance with a tyrosine hydroxylase inhibitor treatment can be much improved by administration of a sustained-release formulation. An important feature of a preferred sustained-release tyrosine hydroxylase inhibitor formulation is the more effective control of free fraction tyrosine hydroxylase inhibitor in serum.

In some embodiments, the pharmaceutical formulation includes an effective amount of one or more another therapeutic agents. Examples of another agent include, for example, but not limited to, an antidepressant (e.g., a selective serotonin reuptake inhibitor (SSRI), a serotonin-norepinephrine reuptake inhibitor (SNRI), a tricyclic antidepressant, sertraline, fluoxetine, paroxetine, venlafaxine), a benzodiazepine, a glucocorticoid, a cannabinoid or a combination thereof. Additional examples of another agent include, for example, but not limited to, vasopressin analog (e.g., desompressin), a neuromodulating agent (e.g., GABA, an agent that potentiates acetylcholine), rivastigmine, or pilocarpine, or similar agents. In a particular embodiment, the one or more another therapeutic agents may be in the form of sustained release agents.

The formulations described herein can be used to treat any suitable mammal, including primates, such as monkeys and humans, horses, cows, cats, dogs, rabbits, and rodents such as rats and mice. In one embodiment, the mammal to be treated is human.

All patents and literature references cited in the present specification are hereby incorporated by reference in their entirety.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

### Example 1

The following formulation method is an example of the preparation of a slow-release α-methyl-para-tyrosine formulation. Wet granulation, extrusion, and fluid-bed drying processes can be utilized to produce sustained-release α-methyl-DL-tyrosine particles or pellets.

To prepare the wet granules, α-methyl-para-tyrosine, microcrystalline cellulose (Avicel PH 102) and methylcellulose (Methocel A15 LV), at the various percentages, can be placed into a high-shear granulator and mixed for 15 minutes. Deionized (DI) water can be added slowly, and the wet granules can be mixed for another 5-10 minutes.

The pellets can then be dried using a fluid bed dryer. The dried pellets can be discharged from the fluid-bed dryer and be sized by passing through different screens.

The dried pellets can then be encapsulated into hard gelatin capsules.

### Example 2

A PLGA copolymer is provided. α-methyl-para-tyrosine can be loaded into the PLGA copolymer. The formulation may be in the form of tablet or capsule.

The formulation described in Example 1 or 2 can be orally administered to a subject.

Serum can be collected and analyzed. The α-methyl-para-tyrosine composition may achieve a therapeutic effect within 2 hrs and maintain therapeutic effect for at least 24 hours in >95% percent of treated patients.

The composition may allow for consistent release of the active agent from the drug delivery vehicle with no more than 25% variation plus an encapsulation efficiency of over 70%. The composition may release the active agent from the drug delivery vehicle with >85% intact over the entire duration of release.

Having described preferred embodiments of the invention, it is to be understood that the invention is not limited to the precise embodiments, and that various changes and modifications may be effected therein by those skilled in the art without departing from the scope or spirit of the invention as defined in the appended claims.

What is claimed is:

1. A controlled-release pharmaceutical formulation comprising a tyrosine hydroxylase inhibitor.

2. The formulation of claim 1 wherein said controlled-release formulation is a sustained-release formulation.

3. The formulation of claim 2 further comprising a retardant excipient configured to modify a dissolution profile of said sustained-release tyrosine hydroxylase inhibitor.

4. The formulation of claim 3, wherein said retardant excipient comprises at least one selected from hydroxypropyl methylcellulose (HPMC), hydroxyethylcellulose, hydroxypropylcellulose (HPC), methylcellulose, ethylcellulose, cellulose acetate butyrate, cellulose acetate phthalate, hydroxypropylmethyl cellulose phthalate, microcrystalline cellulose, corn starch, polyethylene oxide, polyvinyl alcohol (PVA), polyvinylpyrrolidone (PVP), cross-linked PVP, polyvinyl acetate phthalate, polyethylene glycol, zein, poly-DL-lactide-co-glycolide (PLGA), dicalcium phosphate, calcium sulfate, and mixtures thereof.

5. The formulation of claim 1, wherein said tyrosine hydroxylase inhibitor is racemic α-methyl-para-tyrosine.

6. The formulation of claim 1, wherein said tyrosine hydroxylase inhibitor is metyrosine or α-methyl-L-tyrosine.

7. The formulation of claim 1, wherein said tyrosine hydroxylase inhibitor is α-methyl-D-tyrosine.

8. The formulation of claim 1, wherein said tyrosine hydroxylase inhibitor is a tyrosine derivative.

9. The formulation of claim 8, wherein said tyrosine derivative is methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OME HCl, H-3,5-diiodo-tyr-OME HCl, H-D-3,5-diiodo-tyr-OME HCl, H-D-tyr-OME HCl, D-tyrosine methyl ester hydrochloride, D-tyrosine-ome HCl, methyl D-tyrosinate hydrochloride, H-D-tyr-OMe.HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I₂)—OSu, Fmoc-tyr(3-NO₂)—OH, α-methyl-L-tyrosine, α-methyl-D-tyrosine, α-methyl-para-tyrosine, or a combination thereof.

10. The formulation of claim 1, further comprising a filler wherein said filler comprises at least one selected from acetyltriethyl citrate (ATEC), acetyltri-n-butyl citrate (ATBC), aspartame, lactose, alginates, calcium carbonate, carbopol, carrageenan, cellulose, cellulose acetate phthalate, croscarmellose sodium, crospovidone, dextrose, dibutyl sebacate, ethylcellulose, fructose, gellan gum, glyceryl behenate, guar gum, lactose, lauryl lactate, low-substituted hydroxypropyl cellulose (L-HPC), magnesium stearate, maltodextrin, maltose, mannitol, methylcellulose, microcrystalline cellulose, methacrylate, sodium carboxymethylcellulose, polyvinyl acetate phthalate (PVAP), povidone, shellac, sodium starch glycolate, sorbitol, starch, sucrose, triacetin, triethylcitrate, vegetable based fatty acid, xanthan gum, and xylitol.

11. The formulation of claim 1, configured in a dosage form selected from twice daily, once daily, once every two days, once every three days, once every four days, once every five days, once every six days, and once weekly.

12. The formulation of claim 1, wherein said tyrosine hydroxylase inhibitor is present in an amount of 150-500 mg.

13. The formulation of claim 1, wherein said formulation further comprising an effective amount of one or more another therapeutic agents.

14. The formulation of claim 13, wherein said another agent is an antidepressant, a benzodiazepine, a glucocorticoid, a cannabinoid or a combination thereof.

15. The formulation of claim 13, wherein at least one of said one or more another agents is a vasopressin analog.

16. The formulation of claim 15, wherein the vasopressin analog is desompressin.

17. The formulation of claim 13, wherein at least one of said one or more another agents is a neuromodulating agent.

18. The formulation of claim 17, wherein the neuromodulating agent is GABA.

19. The formulation of claim 17, wherein the neuromodulating agent potentiates acetylcholine.

20. The formulation of claim 17, wherein the neuromodulating agent is rivastigmine, or pilocarpine, or similar agents.

21. The formulation of claim 13, wherein said tyrosine hydroxylase inhibitor is racemic α-methyl-para-tyrosine and wherein said one or more another agents comprise desompressin and GABA.

22. The formulation of claim 14, wherein said antidepressant is a selective serotonin reuptake inhibitor (SSRI), a serotonin-norepinephrine reuptake inhibitor (SNRI), a tricyclic antidepressant, or a combination thereof.

23. The formulation of claim 14, wherein said antidepressant is sertraline, fluoxetine, paroxetine, venlafaxine, or a combination thereof.

24. A method of treatment comprising administering the pharmaceutical formulation of claim 1 to a patient in need thereof.

25. A method of making the pharmaceutical formulation of claim 1, comprising intermixing the tyrosine hydroxylase inhibitor with an effective amount of an excipient to form a mixture, and configuring the mixture into a unit dosage form.

26. A controlled-release pharmaceutical formulation comprising: a tyrosine hydroxylase inhibitor dispersed in a wax matrix, wherein said tyrosine hydroxylase inhibitor is α-methyl-DL-tyrosine, α-methyl-D-tyrosine, α-methyl-L-tyrosine, or a combination thereof.

27. A controlled-release pharmaceutical formulation comprising: a tyrosine hydroxylase inhibitor dispersed in polymer matrix, wherein said tyrosine hydroxylase inhibitor is α-methyl-DL-tyrosine, α-methyl-D-tyrosine, α-methyl-L-tyrosine, or a combination thereof.

28. A controlled-release pharmaceutical formulation comprising: a tyrosine hydroxylase inhibitor dispersed in an encapsulated form, wherein said tyrosine hydroxylase inhibitor is α-methyl-DL-tyrosine, α-methyl-D-tyrosine, α-methyl-L-tyrosine, or a combination thereof.
